SEQUENCE LISTING (6 sequences)

SEQ ID NO: 1 - DNA, 18 bp, Danio Rerio
acagagaccgagaggaag

SEQ ID NO: 2 - DNA, 20 bp, Danio rerio
agggtgctggaagaccctat

SEQ ID NO: 3 - DNA, 23 bp, Danio rerio
catgggtgttttaaagtttctcc

SEQ ID NO: 4 - DNA, 22 bp, Danio rerio
cttcaatatttgccgcctttac

SEQ ID NO: 5 - DNA, 21 bp, Danio rerio
ctgggatgatgttctgactgg

SEQ ID NO: 6 - DNA, 21 bp, Danio rerio
agttgtaagcaatgcctcctg

(19) United States
(12) Patent Application Publication
(10) Pub. No.: US 2023/0111558 A1
(43) Pub. Date: Apr. 13, 2023

(54) Composition for Preventing and Treating Heart Dysfunction and its Application Thereof

(71) Applicant: 21ST CENTURY HERBS & HEALTH, Diamond Bar, CA (US)

(72) Inventor: Jianghua LIU, Diamond Bar, CA (US)

(73) Assignee: 21ST CENTURY HERBS & HEALTH

(21) Appl. No.: 17/498,722

(22) Filed: Oct. 11, 2021

(51) Int. Cl. A61K 36/537; A61K 36/258; A61K 31/122; A61P 9/10; A61P 9/00

(52) U.S. Cl. CPC A61K 36/537; A61K 36/258; A61K 31/122; A61P 9/10; A61P 9/00

(57) ABSTRACT

A composition for preventing and treating heart dysfunction and its application thereof, including approximately 6-10% coenzyme Q10, approximately 40-45% Radix salviae Militiorrhizae and approximately 40-45% Radix notoginseng, which is prepared into an oral application such as tablets, capsules, granules or powders in sachets, pills, and oral solution. The extract of Radix salviae Militiorrhizae is a water-soluble extract of the root of Salvia miltiorrhiza Bunge and the powder of Radix notoginseng is a fine powder of the root of Panax notoginseng (Burk.). The composition can be prepared into a dietary supplement for improving heart function or a medicament for treating heart dysfunction by preventing heart failure, treating myocardial injury caused by hypoxia or reperfusion, treating abnormal heartbeat, treating palpitation, relieving pericardial edema, or treating palpitation and chest tightness caused by dysautonomia.

### BACKGROUND OF THE PRESENT INVENTION

### Field of Invention

The present invention relates to a formula of coenzyme Q10, extract of Radix salviae Miltiorrhizae and powder of Radix notoginseng which is capable of preventing and treating heart dysfunction including abnormal heart rate, systolic dysfunction and edema that caused by myocardial injury.

### Description of Related Arts

According to WHO, Cardiovascular diseases (CVDs) are the leading cause of death globally, taking an estimate of 17.9 million lives each year, which is 32% of all deaths worldwide. Coronary artery disease (CAD), a disease of plaque buildup in the blood vessels that supply blood to the heart muscle, is a major type of CVDs.

Myocardial injury can be divided into acute and chronic myocardial injury. It is common in patients without acute coronary syndrome. Acute coronary syndromes can still result in myocardial injury and subsequent myocardial injury despite advances in reperfusion therapy. At present, no guidelines exists for treating patients who have myocardial injury without infarction. Management mainly focused on a thorough evaluation to identify and treat the underlying precipitant. Therefore, prevention and amelioration of myocardial injury is important to reduce mortality of the cardiovascular diseases.

Heart rate, systolic function and edema are three indexes to assess the heart injury. Left ventricular (LV) global systolic function by echocardiography is one of the most common and quickest assessment for heart injury. It can be assessed using changes in the LV dimensions and volumes between LV diastole and systole, which include calculation of ventricles stroke volume (SV), cardiac output (CO), ejection fraction (EF), and fractional area change (FAC). Cardiac edema occurs when the heart's diseased or overworked ventricle isn't able to pump out enough of the blood it receives from the lungs. This causes the heart to hold an excess amount of fluid and resulted in swelling.

Coenzyme Q10 (CoQ10) is an antioxidant that human body produces naturally. The cells use CoQ10 for growth and maintenance. Level of CoQ10 decreases with age. There is growing evidence that CoQ10 is tightly linked to cardiometabolic disorders.

The root of Salvia miltiorrhiza Bunge (Radix salviae Miltiorrhizae, danshen) is a well-known traditional Chinese herb that has been popularly used for treatment of cardiovascular diseases in China and other Asia countries, and widely studied for its myocardial protective effects. The extract of Radix salviae Miltiorrhizae can reduce myocardial infarction area and improve cardiac function in rats with myocardial ischemia reperfusion. Its mechanisms involve scavenging oxygen free radicals, anti-oxidation, inhibiting calcium overload of cardiomyocytes, regulating autophagy, inhibiting inflammatory response, and anti-platelet aggregation. In recent years, the mechanism of bioactive components isolated from Radix salviae Miltiorrhizae in preventing myocardial ischemia reperfusion injury has been revealed continuously. These results provide solid scientific support for Radix salviae Miltiorrhizae in treatment of cardiovascular diseases.

The root of Panax notoginseng (Burk.) (Radix notoginseng, Sanqi) is also widely used to prevent and treat cardiovascular diseases. Studies have shown that saponins isolated Radix notoginseng can effectively improve myocardial tissue damage caused by ischemia/reperfusion, and its mechanism may be related to the regulation of autophagy.

Danshen-Sanqi compound has been used for vascular disease and studies has shown scientific support to this combination use through their synergistic interactions on anti-inflammatory pathways. US patent publication number US20120251634 A1 by Han, et al disclosed the effect of a traditional Chinese drug composition consisting of Danshensu and Notoginsenoside R1 on cardiac microcirculation disorder and myocardial damage induced by ischemia/reperfusion through a study using SD male rats.

Compound Danshen Tablet (CDT) is a popular and well-studied formula that is widely used to improve coronary heart disease, cardiac angina and atherosclerosis in Asia, especially in China. The CDT formula is composed of Radix salviae Miltiorrhizae, Radix notoginseng and Bornelum. Bornelum, called borneol in English, is known to have toxicity, such as causing nausea, vomiting, dizziness, headaches, muscle twitching, and convulsions. In more serious cases, people can slip into a comma or even die.

Compound Danshen Dripping Pills (CDDP) is another Chinese medicine recorded in Chinese pharmacopeia that is composed of Radix salviae Miltiorrhizae, Radix notoginseng and Bornelum, but as a preparation of dripping pills. Modern pharmacological studies have demonstrated that CDDP has the following pharmacologic effects: (1) anti-oxidative, anti-inflammatory, and endothelium-protective effects, inhibition of atherosclerotic plaque formation and neointimal hyperplasia; (2) reduction of myocardial oxygen consumption; improving energy metabolism, and protection of cardiomyocytes; (3) inhibition of platelet adhesion and aggregation; and (4) improvement of microcirculation, which is known to have beneficial effects in the prevention and treatment of ischemic reperfusion injury, injury induced by lipopolysaccharide and other microcirculation disturbances.

Myocardial ischemia, also known as cardiac ischemia, is defined as the deprivation of oxygen and nutrients to the heart. The reperfusion of an ischemic myocardium following a period of ischemia restores blood supply to ischemic tissue, but this is paradoxically associated with further tissue damage, which is triggered via the release of intracellular enzymes and a consequence of reactions.

Zebrafish is useful for modeling human heart diseases due to similarity of zebrafish and mammalian hearts, fast easily tractable development, and readily available genetic methods. Hypoxia-reoxygenation (H/R) treatment has been successfully used to induce heart injury of zebrafish to simulate the model of human myocardial ischemia reperfusion injury. Isoproterenol (ISO), a β-receptor agonist, is also used as a Zebrafish model for heart injury. It can initially increase the force of heart contraction and increase the heart rate, but long-term effects can induce damage to heart muscle cells and eventually lead to heart failure.

Superoxide dismutases (SOD) are a group of metalloenzymes that are found in life. Natural SOD levels in the body drop as the body ages and hence as one age, one becomes more prone to oxidative stress-related diseases. SOD constitute a very important antioxidant defense against oxidative stress in the body and form the front line of defense against reactive oxygen species (ROS)-mediated injury. ROS including O₂ and its reaction product peroxynitrite has a significant role in endothelial and tissue injury associated with ischemia and reperfusion. It is reported that when cardiomyocytes are damaged, the SOD activity is significantly reduced.

Lactate dehydrogenase (LDH) is a specificity enzyme in the cytoplasm that turns sugar into energy. It is found in almost every cell of human body, with highest concentrations in kidney, heart, liver, muscle, lung, and erythrocytes. When these tissues are damaged, they release LDH into the bloodstream or other body fluids. Raised LDH in the blood test may indicate the damage of heart, liver or blood cells. Therefore, LDH has also been used as a marker to evaluate the efficacy of testing samples on tissue injury in animals. But the test can't specify the location of the tissue injured. Reduction of LDH in a test is meaningless.

Atrial natriuretic peptide (ANP) and B-type natriuretic peptide (BNP) are endogenously generated peptides hormone secreted from the cardiac atria and ventricles, respectively. ANP signals in an endocrine and paracrine manner to decrease blood pressure and cardiac hypertrophy. BNP acts locally to reduce ventricular fibrosis. The ANP and BNP gene expressions are markedly augmented in ventricles of patients with a wide variety of cardiovascular diseases.

### SUMMARY OF THE PRESENT INVENTION

Certain variations of the present invention provide a composition which has pharmacological effect on heart dysfunction by regulating the heart rate, reducing pericardial edema and improving heart functions, therefore, preventing and treating cardiac dysfunction and myocardial injury.

In one aspect of the present invention, the composition for preventing and treating cardiac dysfunction and myocardial injury is composed of: Coenzyme Q10 (CoQ10), extract of Radix salviae Militiorrhizae and powder of Radix notoginseng.

Preferably, the ratio of Coenzyme Q10 (CoQ10), extract of Radix salviae Militiorrhizae and powder of Radix notoginseng is 20-60:100-500:100-500 by weight.

The composition can be prepared into an orally administrated capsule, a tablet, a pill, a granule form, a powder form, a liquid form.

Preferably, the composition is formulated into a capsule.

The excipients include binding agents, bulking agents, diluents, tablet-pressing agents, lubricants, disintegrating agents, colorants, flavoring agents, wetting agents, coating agents, suspending agents, emulsifying-agents and etc.

Radix salviae Militiorrhizae is a water-soluble extract. Radix notoginseng is a fine ground powder.

According to the present invention, the composition can significantly regulate the slower or faster heartbeat, improve zebrafish heart function volume (SV), cardiac output (CO), ejection fraction (EF), fractional area change (FAC), and relieve pericardial edema of Zebrafish induced by hypoxia-reoxygenation (H/R) and Isoproterenol (ISO) that simulate human myocardial injury.

These aforesaid pharmacological activities are achieved by regulating excessive gene expression of atrial natriuretic peptide (ANP) and brain natriuretic peptide (BNP).

The composition can be made as a drug or products of dietary supplement for regulate faster and slower heartbeat, heart dysfunction or edema due to myocardial damage caused by ischemia reperfusion, prevent and treat cardiovascular diseases.

In another aspect of the present invention, a method of treating heart dysfunction comprising the step of: administering an effective amount of Coenzyme Q10 (CoQ10), extract of Radix salviae Militiorrhizae and powder of Radix notoginseng to a patient suffering from heart dysfunction.

Preferably, the ratio of Coenzyme Q10 (CoQ10), extract of Radix salviae Militiorrhizae and powder of Radix notoginseng is 20-60:100-500:100-500 by weight.

The composition of Coenzyme Q10 (CoQ10), extract of Radix salviae Militiorrhizae and powder of Radix notoginseng can be prepared into an orally administrated form such as a capsule, a tablet, a pill, a granule form, a powder form, a liquid.

This summary presented above is provided merely to introduce certain concepts and not to identify any key or essential features of the claimed subject matter.

### BRIEF DESCRIPTION OF THE DRAWINGS

FIG. 1 illustrates the effect of Tri-Circulator (TC) on the heart rate (A), stroke volume (B), cardiac output (C), ejection fraction (D), and fractional area change (E) of hypoxia-reoxygenation (H/R) zebrafish. Compared with H/R group, *p<0.05, **p<0.01, ***p<0.001; ^{#}indicates TC vs. CoQ10.

FIG. 2 illustrates the effect of TC and CoQ10 on the ISO-induced zebrafish heart rate (A), stroke volume (B), cardiac output (C), ejection fraction (D), and fractional area change (E). Compared with the ISO group, ***p<0.001.

FIG. 3 illustrates the effect of TC and CoQ10 on ISO-induced zebrafish pericardial edema (A) and pericardial height (B). Compared with the ISO group, ***p<0.001.

FIG. 4 illustrates the effects of TC and CoQ10 on ISO-induced zebrafish SOD (A) and LDH (B). Compared with the ISO group, *p<0.05, **p<0.01.

FIG. 5 illustrates the effects of TC and CoQ10 on ISO-induced zebrafish myocardial hypertrophy genes brain natriuretic peptide (BNP) (A) and atrial natriuretic peptide (ANP) (B). Compared with the ISO group, ***p<0.001, Indicates TC vs. Q10.

### DETAILED DESCRIPTION OF THE PREFERRED EMBODIMENT

The following detailed description of the preferred embodiments is the preferred mode of carrying out the invention. The description is not to be taken in any limiting sense. It is presented for the purpose of illustrating the general principles of the present invention.

A composition for preventing and treating cardiac dysfunction and myocardial injury according to a preferred embodiment of the present invention is composed of: Coenzyme Q10 (CoQ10), extract of Radix salviae Militiorrhizae and powder of Radix notoginseng. The composition has pharmacological effect on heart dysfunction by regulating the heart rate, reducing pericardial edema and improving heart functions, therefore, preventing and treating cardiac dysfunction and myocardial injury.

Preferably, the ratio of Coenzyme Q10 (CoQ10), extract of Radix salviae Militiorrhizae and powder of Radix notoginseng is 20-60:100-500:100-500 by weight.

Radix salviae Militiorrhizae is a water-soluble extract. Radix notoginseng is a fine ground powder.

The composition can be prepared into an orally administrated form such as a capsule, a tablet, a pill, granules, powder and liquid.

According to another aspect of the present invention, a method of treating heart dysfunction comprises the step of: administering an effective amount of Coenzyme Q10 (CoQ10), extract of Radix salviae Militiorrhizae and powder of Radix notoginseng to a patient suffering from heart dysfunction.

Preferably, the ratio of Coenzyme Q10 (CoQ10), extract of Radix salviae Militiorrhizae and powder of Radix notoginseng is 20-60:100-500:100-500 by weight.

The composition of Coenzyme Q10 (CoQ10), extract of Radix salviae Militiorrhizae and powder of Radix notoginseng can be prepared into an orally administrated form such as a capsule, a tablet, a pill, a granule form, a powder form, a liquid.

The CoQ10, Radix salviae Militiorrhizae and Radix notoginseng, when used as one composition, exhibit a unique properties and treatment effect different from CoQ10 or a mixture of Radix salviae Militiorrhizae and Radix notoginseng.

### Experiment One

### Experimental Materials

Coenzyme Q10 (CoQ10) is purchased from Meilun Biotechnology Co., Ltd. (Dalian, China). The composition, namely the Tri-Circulator (TC) capsule, is prepared by 21^{st} Century Herbs & Health Inc, USA. Each TC capsule contains 40 mg of CoQ10 and 450 mg proprietary blend of extract of Radix salviae Miltiorrhizae and powder of Radix notoginseng (the ratio of weight of extract of Radix salviae Miltiorrhizae and powder of Radix notoginseng is 1:1).

### Experimental Animals

The zebrafish experiments are conducted in accordance to the internationally accepted standards. The wild type zebrafish strain AB is provided by the Developmental Biology Platform of Chongqing Medical University. Using the Shanghai Haisheng zebrafish aquatic ecosystem, the temperature in the fish tank is 28.5° C., the water temperature is 28-30° C., the salinity is 0.03%-0.04%, the pH is 7.2-7.6, and the photoperiod is 14 h light-10 h dark.

Establishment of H/R Model for Zebrafish

Three-day post fertilization wild-type of zebrafish with normal development are used for the experiment. The zebrafish are divided into 4 groups, namely the control group, the H/R (Hypoxia-reoxygenation) group, the TC group (CoQ10=4.0 mg/L), and the CoQ10 group (CoQ10=4.0 mg/L), n=30. Fill the beaker with circulating water for fish breeding, and then fill the water with high purity nitrogen. At the same time, use dissolved oxygen meter to detect the oxygen concentration in the water. Stop filling nitrogen until the oxygen concentration is stable at 0.00 mg/L. The air in the bevel glass bottle is discharged with nitrogen, and the prepared hypoxic water is put into the bevel glass bottle. The juvenile zebrafish of 3 days are put into the bottle, the cap is tightened, the glass bottle is placed into a plastic sealed bag, and then the sealed bag is vacuumed to ensure that no air enters. The end point of hypoxia is that the young fish sank to the bottom and remained motionless for 1 minute. At the same time, the zebrafish bent its body, turned the glass bottle upside down and then put it flat. The zebrafish sank head down and vertically. Then, the zebrafish are taken out and put into aquaculture water or medicine solution prepared with aquaculture water. After reoxygenation for 24 hours, the heart function indexes of zebrafish are examined.

ISO-Induced Model of Zebrafish Myocardial Injury

Three-day post fertilization wild-type zebrafish with normal development are used for the experiment and divided them into 4 groups, namely the control group, the ISO group (ISO=0.02 mg/L), the TC group (CoQ10=4.0 mg/L), and the CoQ10 group (CoQ10=4.0 mg/L), 30 in each group; The zebrafish in the ISO group are immersed in the ISO solution prepared with embryo culture water for 24 hours, then taken out and recorded their heart rate under a microscope. The signs of successful ISO-induced myocardial injury in zebrafish are a significant decrease in heart rate and the formation of pericardial edema. The zebrafish in the TC and CoQ10 groups are respectively immersed in embryo breeding water containing ISO and TC or CoQ10, incubated for 24 hours and then taken out for photography under a microscope.

First, the zebrafish are placed at room temperature and counted the number of heart beats within 15 seconds under a microscope. Then, the heart rate is obtained by multiplying it by 4. Then the zebrafish are placed in a petri dish, fixed with 1% low-melting agar, and held in a lateral position (with the eyes on top of the segments and the tail at the same level as the body). Fifty-eight images are generated by using an inverted fluorescence microscope for five consecutive shots. The diastolic and systolic images are selected, and the length (a) and width (b) of the ventricles are measured. The volume (V) of the ventricles is calculated with the ellipsoidal volume formula, and the surface area (S) of the ventricles is calculated with the elliptical area formula. The stroke-volume (SV), ejection fraction (EF), and fractional area change (FAC), cardiac output (CO) of zebrafish are calculated. Ventricular volume (V)=4/3π(a/2) (b/2)²; Stroke-volume (SV)=V_{diastolic}−V_{systolic}; Ejection Fraction (EF)=(V_{diastole}−V_{systole})/V_{diastole}; Ventricular surface area (S)=(a/2) (b/2)π; Fractional Area Change (FAC)=(S_{diastolic}−S_{systolic})/S_{diastolic}; Cardiac Output (CO)=Cardiac volume*heart rate.

Measurement of Cardiac Edema in Zebrafish Induced by ISO

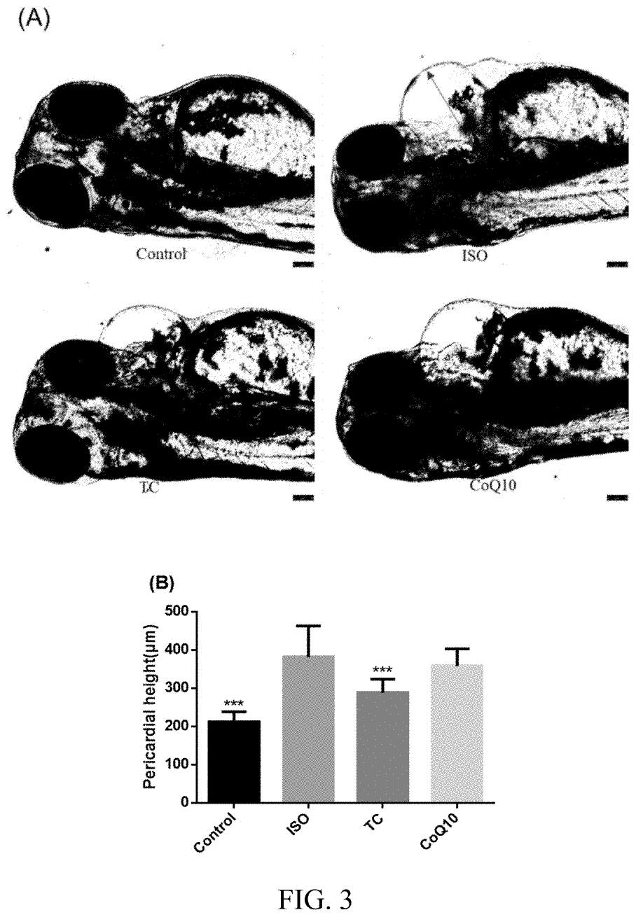

Images are used to measure the height of the pericardium, the distance between the front of the yolk sac and the top of the pericardium, as shown by the arrow in FIG. 3(A).

Gene Expression Detected by RT-PCR

Zebrafish RNA extraction, reverse transcription and quantitative amplification are performed in accordance with the instructions of the kit obtained from Nanjing Vazyme Biotech Co., Ltd, the article numbers are RC101, R223-01 and Q711-02.

Primer Sequence:

ANP:
Forward (SEQ ID NO. 1):
ACAGAGACCGAGAGGAAG;
Reverse (SEQ ID NO. 2):
AGGGTGCTGGAAGACCCTAT.
BNP:
Forward (SEQ ID NO. 3):
CATGGGTGTTTTAAAGTTTCTCC;
Reverse (SEQ ID NO. 4):
CTTCAATATTTGCCGCCTTTAC.
GAPDH:
Forward (SEQ ID NO. 5):
CTGGGATGATGTTCTGACTGG;
Reverse (SEQ ID NO. 6):
AGTTGTAAGCAATGCCTCCTG.

ANP refers to atrial natriuretic peptide, BNP refers to brain natriuretic peptide, GAPDH refers to glyceraldehyde 3-phosphate dehydrogenase.

Measurement of Superoxide Dismutase (SOD), Lactate Dehydrogenase (LDH)

The detection of SOD and LDH are carried out according to the instructions of the kits, which are purchased from NanJing JianCheng Bioengineering Institute with the article numbers of A001-3, A020-2 and A019-2-1, respectively.

Statistical Analysis

SPSS20.0 statistical software is used for data processing, and the experimental results are expressed by means±SD. Single factor is used to compare the mean of multiple groups of samples. P<0.05 indicated a statistical difference.

Results

Effects of TC and CoQ10 on Cardiac Function of H/R Zebrafish

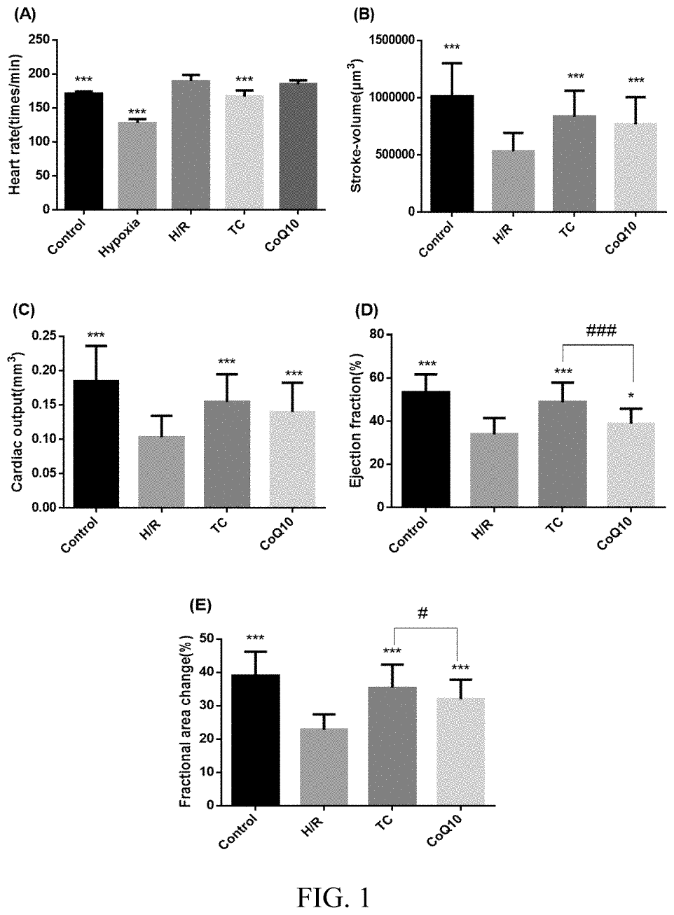

The heart rate of zebrafish increased significantly after H/R treatment (see FIG. 1A). TC could significantly reduce the increased heart rate of zebrafish with H/R treatment closely back to normal. But CoQ10 at the testing concentration has no significant effect. After H/R treatment, zebrafish's SV, CO, EF, and FAC all decreased significantly, and both TC and CoQ10 could significantly increase the reduced number of SV, CO, EF, and FAC toward to normal (see FIGS. 1B-1E). Furthermore, TC has more significant effect than CoQ10 on EF (see FIG. 1D) and FAC (see FIG. 1E).

Effects of TC and CoQ10 on the Cardiac Function of Zebrafish Induced by ISO

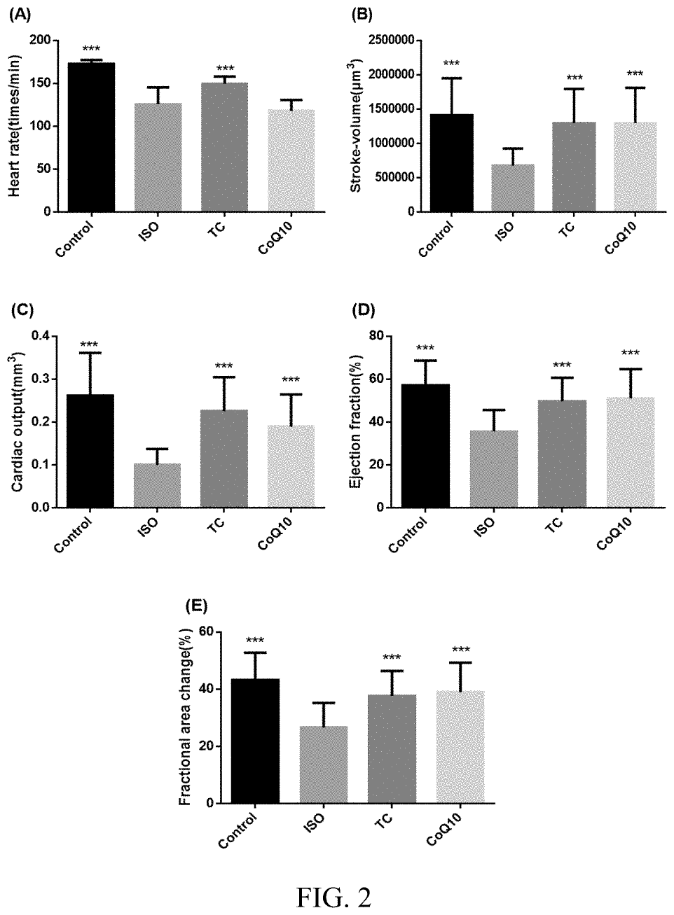

The zebrafish's heart rate, CO, SV, EF, and FAC all decreased significantly after ISO induction (see FIG. 2). TC can significantly increase the decreased heart rate induced by ISO, but CoQ10 at the testing concentration has no significant effect (see FIG. 2A). TC and CoQ10 can significantly increase the decreased number of SV (see FIG. 2B), CO (see FIG. 2C), EF (see FIG. 2D), and FAC (see FIG. 2E).

Effects of TC and CoQ10 on ISO-Induced Zebrafish Pericardial Edema

The zebrafish pericardium increased significantly after ISO induction (see FIG. 3A). The pericardial height is used to quantify the edema. The results showed that TC has a significant alleviating effect on pericardial edema induced by ISO, but CoQ10 has no effect at the testing concentration (see FIG. 3B).

The Effects of TC and CoQ10 on ISO-Induced Biochemical Indexes of Zebrafish

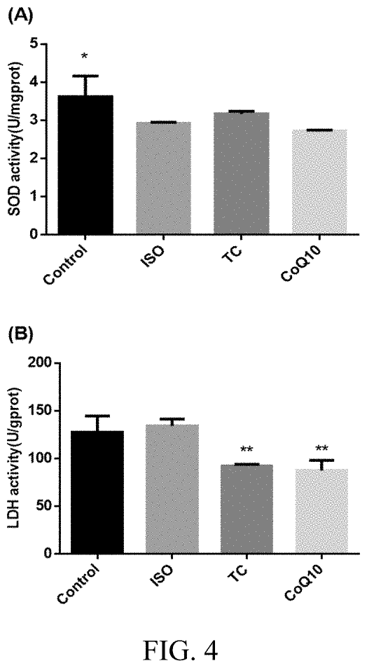

The results showed that ISO significantly reduced the SOD level in comparison with the control group. But TC and CoQ10 have no significantly effect on the decreased activity of SOD reduced by ISO (see FIG. 4A). ISO does not cause a significant increase in LDH (see FIG. 4B). Although TC and CoQ10 reduced the activity of LDH, the result is insignificant.

Effects of TC and CoQ10 on Gene Expression of ANP and BNP in ISO-Induced Zebrafish

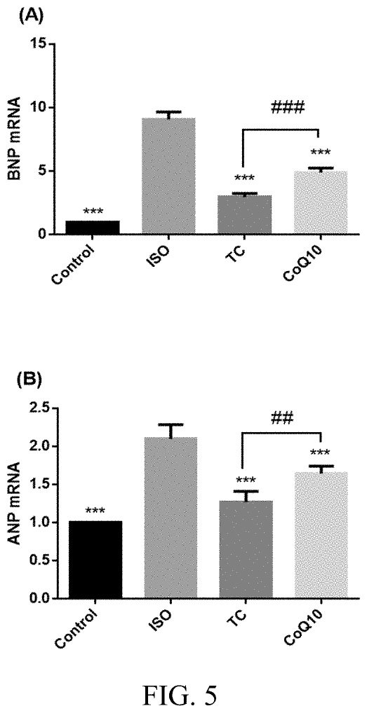

The results show that ISO significantly stimulated the gene excessive express of BNP and ANP, TC and CoQ10 can significantly reduce the excessive expression of BNP and ANP (see FIG. 5). Furthermore, TC has more significant effect on both genes than CoQ10.

In summary, both TC and CoQ10 have significant effect on stroke-volume (SV), cardiac output (CO), ejection fraction (EF), and fractional area change (FAC), Fractional Area Change (FAC), and gene expression of ANP and BNP. Moreover, TC has more significant effect than CoQ10 on EF, FAC, and gene expression of ANP and BNP, indicating that both CoQ10 and the blend of two herbs make their contribution to the efficacy. In addition, TC has significant effect on adjusting heart rate and alleviating effect on pericardial edema while CoQ10 has no significant effect on adjusting heart rate and alleviating pericardial edema.

In other words, even though both TC and CoQ10 groups contain the same amount of CoQ10, their effect on heart injury are different. This indicates that not only CoQ10, but also the proprietary blend of extract of Radix salviae Miltiorrhizae and powder of Radix notoginseng make contribution for TC's effect on recovery of the induced myocardial injury. And the proprietary blend plays an important role on TC's effect regulating heart rate and alleviating effect on pericardial edema.

It is worth mentioning that although Experiment One does not directly compare the effect of TC and Danshen-Sanqi, it is reasonable predictable that Danshen-Sanqi cannot achieve the effect of TC. The synergetic effect of danshen-sanqi and CoQ10, which is not reasonably predictable or suggested by studies on Danshen-Sanqi alone or studies on CoQ10 alone, is the fundamental elements of the present invention.

Case Studies

The Tri-Circulation (TC) capsules have been given to human objects with heart dysfunction. The administration is two capsules each time, twice per day. The human objects have testified that the TC composition is safe to use and is capable of providing a treatment effect. In this case series studies involving about 300 of human objects, the TC composition can effectively provide relieve to symptoms of chest pain or tightness, short of breath, abnormal heartbeat, palpitation and weakness.

Case 1: Patient 1, Female, Age 90

Symptoms: Diagnosed coronary artery disease by cardiologist. Coronary arteries were blocked 70%. The specialist suggested surgery, the patient refused because of age and health concerns (one of her leg was cut because of tumor when she was young). She had to use oxygen tube. The heartbeat was 120/min for serval days before the family called for acupuncture treatment when she lost consciousness at home. The face color looked like a white paper and the pulse was too weak to feel with fingers.

Treatment: Right after the first acupuncture treatment, the patient waked up. Then she took a course of Tri-Circulator. Two capsules each time, twice per day (morning and evening).

Result: The heartbeat became more strength and dropped to 75 after administrated TC for 30 days. No adverse effect is observed.

Case 2: Patient 2, Female, Age 62

Symptoms: Diagnosed coronary artery disease by cardiologist. She experienced hort of breath, fatigue, poor sleep. The prescribed medicine did not help.

Treatment: She took a course of Tri-Circulator. Two capsules each time, twice per day (morning and evening).

Result: She feels more comfortable after taking TC for 30 days. All symptoms are disappeared after 60 days. No adverse effect is observed.

Case 3: Patient 3, Male, Age 58

Symptoms: Heartbeat 130/min, fatigue, anxiety, had panic heart attack history. No abnormal was found in heart examination.

Treatment: He took a course of Tri-Circulator. Two capsules each time, twice per day (morning and evening).

Result: The heartbeat dropped to 80/min after taking TC for 60 days. He feels that his energy is gained back. He no longer feels fatigue. No adverse effect is observed.

Case 4: Patient 4, Male, Age 53

Symptoms: Diagnosed coronary artery disease by cardiologist. Chest tightness and fatigue for 1 year, occasionally short of breath, DL and TG were higher.

Treatment: He took a course of Tri-Circulator. Two capsules each time, twice per day (morning and evening).

Result: Symptoms of chest tightness and fatigue are reduced significantly after taking TC for 30 days. All symptoms are disappeared after 90 days. No adverse effect is observed.

Case 5: Patient 5, Male, Age 41

Symptoms: Frequent pain and tightness around the heart area, palpitation and anxiety sometimes. Did not see cardiologist and refuse prescribed medicine. Prefer natural therapy.

Treatment: He took a course of Tri-Circulator. Two capsules each time, twice per day (morning and evening).

Result: All symptoms are disappeared after taking TC for 30 days. He continues to take another two bottles (60 days) and feels well. No adverse effect is observed.

The above case studies indicated that it is safe and effective to take the composition of the present invention for heart treatment.

### Preferred Embodiment 1

40 mg of CoQ10, 250 mg water extract of Radix salviae Miltiorrhizae and 250 mg powder of Radix notoginseng are well mixed to prepare a composition of the present invention, which is used as a dietary supplement for improving heart condition.

### Preferred Embodiment 2

50 mg of CoQ10, 250 mg water extract of Radix salviae Miltiorrhizae and 250 mg powder of Radix notoginseng are well mixed to prepare a composition of the present invention, which is used as a dietary supplement for improving heart condition.

### Preferred Embodiment 3

45 mg of CoQ10, 250 mg water extract of Radix salviae Miltiorrhizae and 250 mg powder of Radix notoginseng are well mixed to prepare a composition of the present invention, which is used as botanical drug composition.

### Preferred Embodiment 4

50 mg of CoQ10, 250 mg water extract of Radix salviae Miltiorrhizae and 250 mg powder of Radix notoginseng are well mixed to prepare a composition of the present invention, which is used as botanical drug composition.

### Preferred Embodiment 5

This preferred embodiment is the same the preferred embodiment 1-4 except that: sodium carboxymethyl cellulose and magnesium stearate are added and well mixed with the CoQ10, the extract of Radix salviae Miltiorrhizae and the powder of Radix notoginseng, then fill into capsules with capsule filler machine.

### Preferred Embodiment 6

This preferred embodiment is the same the preferred embodiment 1-4 except that: 1 wt % crospovidone, 0.2 wt % magnesium stearate, and 10 wt % microcrystalline cellulose (said weight percentage is the percentage of the weight of each individual vehicle to the total weight of all of the raw materials) are added to the CoQ10, the extract of Radix salviae Miltiorrhizae and the powder of Radix notoginseng, and a soft material is prepared by using an ethanol solution, granulated and air-dried at 60° C. to give granules. The granules are sifted and pressed into tablets, thereby orally disintegrating tablets are obtained.

### Preferred Embodiment 7

This preferred embodiment is the same the preferred embodiment 1-4 except that: 1 weight part of the composition of the present invention, 1 weight parts of sucrose powder and 1 weight parts of dextrin are granulated, and the granulates are pressed into blocks and dried to produce granules.

### Preferred Embodiment 8

This preferred embodiment is the same the preferred embodiment 1-4 except that: 1 weight part of the composition of the composition of the present invention, 1 weight parts of sucrose, 1 weight parts of dextrin and a proper amount of ethanol are used to give granules, the obtained granules are dried and sifted.

### Preferred Embodiment 9

This preferred embodiment is the same the preferred embodiment 1-4 except that: make the mixture into pill with water pill machine.

### Preferred Embodiment 9

The present invention, while illustrated and described in terms of a preferred embodiment and several alternatives, is not limited to the particular description contained in this specification. Additional alternative or equivalent components could also be used to practice the present invention.

What is claimed is:

1. A composition for preventing and treating heart function, comprising coenzyme Q10, extract of Radix salviae Militiorrhizae and powder of Radix notoginseng.

2. The composition, as recited in claim 1, comprising, by percentage weight, approximately 6-10% coenzyme Q10, approximately 40-45% extract of Radix salviae Militiorrhizae and approximately 40-45% powder of Radix notoginseng.

3. The composition, as recited in claim 2, wherein said extract of Radix salviae Militiorrhizae is a water-soluble extract of the root of salviae Militiorrhizae Bunge.

4. The composition, as recited in claim 2, wherein said powder of Radix notoginseng is a fine powder of the root of Panax notoginseng (Burk.).

5. The composition, as recited in claim 3, wherein said powder of Radix notoginseng is a fine powder of the root of Panax notoginseng (Burk.).

6. The composition, as recited in claim 2, is prepared into an orally administered form consisting of the group selecting from tablet, capsule, granule, powder in sachet, pill, and solution.

7. The composition, as recited in claim 3, is prepared into an orally administered form consisting of the group selecting from tablet, capsule, granule, powder in sachet, pill, and solution.

8. The composition, as recited in claim 6, is used as a dietary supplement for improving heart function.

9. The composition, as recited in claim 7, is used as a dietary supplement for improving heart function.

10. The composition, as recited in claim 6, is used as a medicament for human object for preventing heart dysfunction, treating myocardial injury caused by hypoxia or reperfusion, treating abnormal heartbeat, treating palpitation, relieving pericardial edema, or treating palpitation and chest tightness caused by dysautonomia.

11. The composition, as recited in claim 7, is used as a medicament for human object for preventing heart dysfunction, treating myocardial injury caused by hypoxia or reperfusion, treating abnormal heartbeat, treating palpitation, relieving pericardial edema, or treating palpitation and chest tightness caused by dysautonomia.

12. A method of improving heart function, comprising the step of: administering an effect amount of a composition comprising: coenzyme Q10, extract of Radix salviae Militiorrhizae and powder of Radix notoginseng to a human object suffering from heart dysfunction.

13. The method, as recited in claim 12, wherein the composition comprises, by percentage weight, approximately 6-10% coenzyme Q10, approximately 40-45% Radix salviae Militiorrhizae and approximately 40-45% Radix notoginseng.

14. The method, as recited in claim 13, wherein the extract of Radix salviae Militiorrhizae is a water-soluble extract of the root of salviae Militiorrhizae Bunge; and the powder of Radix notoginseng is a fine powder of the root of Panax notoginseng (Burk.).

15. The method, as recited in claim 13, wherein the composition is prepared into an orally administered form consisting the group of tablet, capsule, granule, powder in sachet, pill, and solution.

16. The method, as recited in claim 15, wherein the heart function is improved by preventing heart dysfunction, treating myocardial injury caused by hypoxia or reperfusion, treating abnormal heartbeat, treating palpitation, relieving pericardial edema, or treating palpitation and chest tightness caused by dysautonomia.
